(19) United States
(12) Patent Application Publication
(10) Pub. No.: US 2016/0360951 A1
(43) Pub. Date: Dec. 15, 2016

(54) BEND SHAPE ESTIMATION SYSTEM, TUBULAR INSERTION SYSTEM, AND BEND SHAPE ESTIMATION METHOD OF BEND MEMBER

(71) Applicant: OLYMPUS CORPORATION, Tokyo (JP)

(72) Inventor: Jun HANE, Tokyo (JP)

(73) Assignee: OLYMPUS CORPORATION, Tokyo (JP)

(21) Appl. No.: 15/248,650

(22) Filed: Aug. 26, 2016

Related U.S. Application Data
(63) Continuation of application No. PCT/JP2015/057873, filed on Mar. 17, 2015.

(30) Foreign Application Priority Data
Mar. 24, 2014 (JP) 2014-059552

(51) Int. Cl. A61B 1/005; A61B 1/04; G01B 11/24; A61B 1/06; G02B 23/24; A61B 34/20; A61B 1/00

(52) U.S. Cl. CPC A61B 1/0057; A61B 34/20; A61B 1/04; A61B 1/00009; A61B 1/00045; A61B 1/0661; A61B 1/0052; G02B 23/2484; G01B 11/24; A61B 2034/2061

(57) ABSTRACT

When a predetermined range of a bend member is divided into a plurality of segments which neighbor in order in a longitudinal direction and are estimation units each having at least information of a length, a curvature and a direction for estimating a bend shape of the bend member, a shape estimation unit includes a segment shape estimation unit which estimates a shape of each of the segments by using segment information including at least one piece of curvature information with respect to each segment, and a bend member shape estimation unit which estimates the bend shape in the predetermined range by connecting end portions of each two neighboring segments, such that tangent directions of end portions of the shapes of the two neighboring segments coincide and directions around the tangent directions coincide.

### CROSS-REFERENCE TO RELATED APPLICATIONS

This application is a Continuation application of PCT Application No. PCT/JP2015/057873, filed Mar. 17, 2015 and based upon and claiming the benefit of priority from the prior Japanese Patent Application No. 2014-059552, filed Mar. 24, 2014, the entire contents of which are incorporated herein by reference.

### BACKGROUND OF THE INVENTION

1. Field of the Invention

The present invention relates to a bend shape estimation system which estimates a bend shape in a predetermined range of a bend member, and a bend shape estimation method of the bend member.

Furthermore, the present invention relates to a tubular insertion system for performing observation by being inserted in a lumen, or for performing treatment such as repair, therapeutic treatment or sampling, the tubular insertion system being represented by a flexible endoscope or a catheter, and the above-described bend shape estimation system being applied to the tubular insertion system. Specifically, the invention relates to a tubular insertion system, at least a part in a longitudinal direction of which has a variable shape, such as a medical endoscope (e.g. an upper gastrointestinal endoscope, a colonoscope, an ultrasonic endoscope, etc.), an industrial endoscope, a rigid endoscope with a partial bend mechanism, a manipulator (robot arm), or a catheter.

2. Description of the Related Art

As regards a device such as an endoscope, which is inserted in a body cavity, if the device is once inserted in the body, an operator such as a doctor cannot confirm, directly by the eye, the state of the device. Thus, the relationship between the up-and-down/right-and-left direction of an observation site or an image, which the operator is viewing, and the disposition of an internal organ or the like becomes uncertain, and, in some cases, the operator mistakes the direction of insertion or the direction of bending. In addition, there is concern that a load may act on the internal organ, due to the operator performing a mistaken operation, or, without the operator being aware of it.

To address these problems, such an attempt has been made that a bend amount sensor is assembled in an insertion section of an endoscope, the curvature or bend amount of the insertion section of the endoscope is detected at a plurality of points of the insertion section, and the shape of the insertion section is detected, thereby to improve the insertion and operation of the insertion section by the operator.

For example, Jpn. Pat. Appln. KOKAI Publication No. 2007-044412 (hereinafter referred to as patent document 1) discloses an endoscope insertion shape detection probe which detects the bend state at a plurality of detection points of an insertion section of an endoscope, and reproduces the bend shape of the insertion section from the information of the detected bend state. Specifically, in this patent document 1, by utilizing the detected angles which were detected at the respective detection points, the respective detection points are connected by angled lines on the basis of the distances between the respective detection points. Thereby, the bend shape of the insertion section is shown.

An operator such as a doctor performs a safe and easy insertion operation, based on various information pieces such as the shape state of the endoscope insertion section.

### BRIEF SUMMARY OF THE INVENTION

According to one aspect of the present invention, there is provided a bend shape estimation system a bend shape estimation system including a shape estimation unit, wherein when a predetermined range of a bend member is divided into a plurality of segments which neighbor in order in a longitudinal direction and are estimation units each having at least information of a length, a curvature and a direction for estimating a bend shape of the bend member, the shape estimation unit includes a segment shape estimation unit configured to estimate a shape of each of the segments by using segment information including at least one piece of curvature information with respect to each segment, and a bend member shape estimation unit configured to estimate the bend shape in the predetermined range of the bend member by connecting end portions of each two neighboring segments, such that tangent directions of end portions of the estimated shapes of the two neighboring segments coincide and directions around the tangent directions coincide.

According to another aspect of the present invention, there is provided a tubular insertion system a tubular insertion system including an insertion section with flexibility configured to be inserted in a lumen of a subject and to perform a predetermined work, the bend shape estimation system according to the one aspect for estimating a bend shape of the insertion section, the insertion section being the bend member, and a shape sensor including, in each segment, at least one sensing part for acquiring the segment information of each segment.

According to further aspect of the present invention, there is provided a bend shape estimation method of a bend member in a bend shape estimation system such that a predetermined range with flexibility of the bend member is divided into a plurality of segments which neighbor in order in a longitudinal direction, a shape of each of the segments is estimated by using segment information including at least one piece of curvature information with respect to each segment, and a shape of the bend member is estimated based on each estimated segment shape, the method including segmenting the predetermined range of the bend member into a plurality of segments neighboring in order in a longitudinal direction of the bend member, such that at least one of a plurality of sensing parts, which are disposed along the longitudinal direction of the bend member, is included in each of the segments, acquiring segmentation information which is information necessary for shape estimation, other than curvature information, the segmentation information including a disposition and a length of each segment, acquiring segment information which includes the curvature information detected at the plurality of sensing parts, estimating a segment shape including at least one of a curvature, a bend amount, a bend direction and a bend shape of each segment, based on the segmentation information and the segment information, connecting neighboring segments whose segment shapes were estimated and estimating a shape of an entirety of the predetermined range of the bend member, confirming whether the shape estimation is continued or not, repeating the acquiring segment information, the estimating segment shape and the connecting neighboring segments and estimating the shape if a result of the confirming is that the shape estimation is continued, and exiting the repeating and finishing the shape estimation if the result of the confirming is that the shape estimation is finished.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a view illustrating the configuration of an endoscope system as a tubular insertion system according to an embodiment of the present invention.

FIG. 2 is a view illustrating the configuration of a sensor in a case of using a fiber sensor.

FIG. 3A is a view for explaining a detection principle of the fiber sensor, and illustrates a case in which an optical fiber is not bent.

FIG. 3B is a view for explaining the detection principle of the fiber sensor, and illustrates a case in which the optical fiber is bent upward on the sheet of the drawing.

FIG. 3C is a view for explaining the detection principle of the fiber sensor, and illustrates a case in which the optical fiber is bent downward on the sheet of the drawing.

FIG. 4 is a block configuration diagram of a bend shape estimation system according to an embodiment of the present invention.

FIG. 5 is a view illustrating an arrangement of sensing parts for direction detection, and illustrates a case in which two sensing parts are arranged at an identical position of the optical fiber.

FIG. 6 is a view for describing a coordinate system for a calculation of direction detection.

FIG. 7 is a view illustrating an arrangement of sensing parts for direction detection, and illustrates a case in which two sensing parts are slightly spaced apart and arranged on the optical fiber.

FIG. 8 is a view for explaining a calculation method of direction detection, and illustrates an example of detection of a bend amount and a bend direction.

FIG. 9 is a view illustrating a concrete example of a bend and a detected shape.

FIG. 10 is a view for describing segmentation, and illustrates a state before segmentation.

FIG. 11 is a view for describing segmentation, and illustrates a state after segmentation.

FIG. 12 is a view illustrating an example of the connection of segment bend shapes.

FIG. 13 is a view illustrating segmentation in a case in which a segment boundary is set at a midpoint between two sensing parts.

FIG. 14A is a view illustrating segmentation in a case in which a segment boundary is set at a change point of bend characteristics.

FIG. 14B is an enlarged view of a vicinity of the segment boundary in FIG. 14A.

FIG. 15 is a view illustrating segmentation in a case in which a segment boundary is set at connection portion to a rigid body.

FIG. 16 is a view illustrating segmentation in a case in which a segment boundary is determined based on bend amounts.

FIG. 17 is a view showing a table for explaining segmentation in a case in which a segment boundary is determined based on a ratio of minimum R in use, or an order of minimum R in use.

FIG. 18 is a view showing a table for explaining segmentation in a case in which a segment boundary is determined based on a ratio of flexural rigidities EI, or an order of flexural rigidities EI.

FIG. 19 is a view for describing segmentation in a case in which segmentation is implemented such that a plurality of pairs of sensing parts are included.

FIG. 20 is a flowchart for describing a bend shape estimation method.

### DETAILED DESCRIPTION OF THE INVENTION

An embodiment for implementing the present invention will be described hereinafter with reference to the accompanying drawings.

Incidentally, in the description below, although a medical endoscope is described by way of example, the present invention is generally applicable to insertion systems if such insertion systems perform insertion and treatment by operating insertion sections. For example, the invention is applicable to a catheter, a manipulator, and an industrial endoscope, as well as a medical endoscope (e.g. an upper gastrointestinal endoscope, a colonoscope, an ultrasonic endoscope, a cystoscope, a pyeloscope, a bronchoscope, etc.).

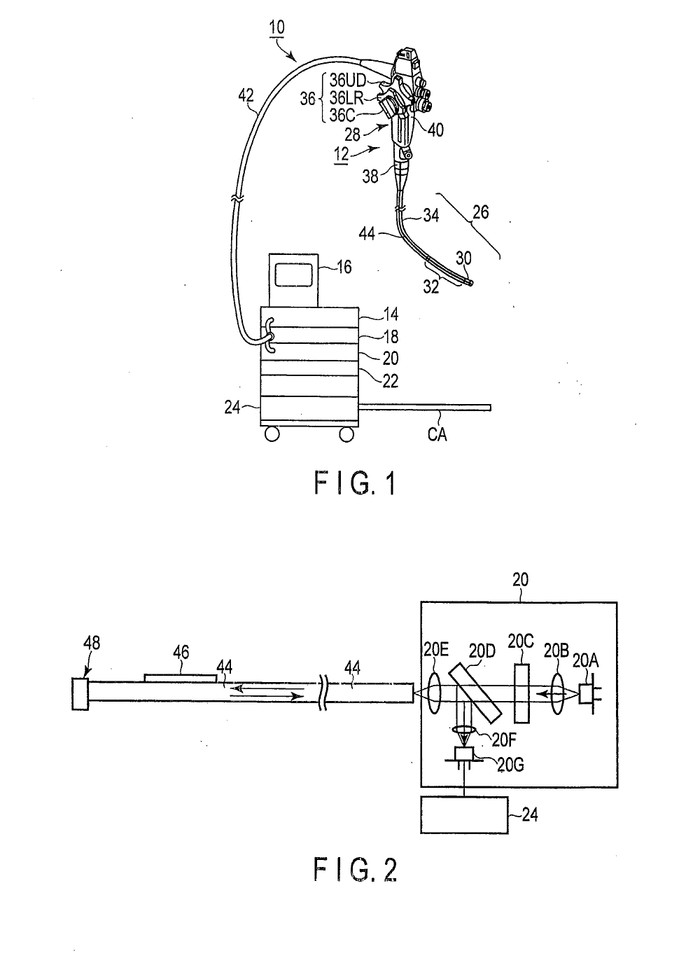

As illustrated in FIG. 1, an endoscope system 10, as a tubular insertion system according to an embodiment of the present invention, includes an endoscope 12, an image processing device (video processor) 14, and a display unit (monitor) 16. The endoscope 12 acquires image of an observation target. The image processing device 14 image-processes to an acquisition result of the endoscope 12. The display unit 16 is connected to the image processing device 14, and displays an observation image which was acquired by the endoscope 12 and image-processed by the image processing device 14.

The endoscope system 10 further includes a light source device 18, a light emission/detection device 20, a control device 22, and a shape estimation unit 24. The light source device 18 emits illumination light toward the endoscope 12. The light emission/detection device 20 emits light for detection of a shape sensor (to be described later), which is different from the illumination light, and detects this light. The control device 22 controls the endoscope system 10. Based on the detection result of the light emission/detection device 20, the shape estimation unit 24 estimates a bend shape in a predetermined range of a bend member to which the shape sensor (to be described later) is attached. The shape estimation unit 24 may be configured by a hardware circuit or a processor. If the shape estimation unit 24 is configured by the processor, a program codes for operating the processor as the shape estimation unit 24 when the processor executes it are stored in a processor internal memory or an external memory accessible by the processor. The shape estimation unit 24 is connected to a display unit (not shown) via a cable CA, and can display and output the estimated bend shape on this display unit. Alternatively, the shape estimation unit 24 is connected to a network communication unit (not shown) via the cable CA, and can transmit and output the estimated bend shape to some other device by the network communication unit via a network such as a LAN or the Internet.

Here, the observation target is, for instance, an affected part or a lesion part in a subject (e.g. body cavity (lumen)).

In the endoscope 12, there are provided an elongated insertion section 26 that is a bend member, and an operation section 28 that is coupled to a proximal end portion of the insertion section 26. The endoscope 12 is a tubular insertion apparatus configured to insert the tubular insertion section 26 into a body cavity.

The insertion section 26 includes a distal-end rigid portion 30, a bend portion 32 which bends, and a flexible tube portion 34, from the distal-end side toward the proximal-end side of the insertion section 26. Here, a proximal end portion of the distal-end rigid portion 30 is coupled to a distal end portion of the bend portion 32, and a proximal end portion of the bend portion 32 is coupled to a distal end portion of the flexible tube portion 34.

The distal-end rigid portion 30 is a distal end portion of the insertion section 26 and a distal end portion of the endoscope 12, and is a rigid member.

The bend portion 32 bends in a desired direction in accordance with an operation of the bend operation unit 36 provided on the operation section 28 by the operator (a worker such as a doctor) of the endoscope 12. By operating the bend operation portion 36, the operator bends the bend portion 32. By the bending of the bend portion 32, the position and direction of the distal-end rigid portion 30 are varied, and the observation target is captured in an observation view field. Illumination light from the light source device 18 is radiated on the captured observation target, and the observation target is illuminated. The bend portion 32 is constituted by coupling a plurality of node rings (not shown) along a longitudinal axis direction of the insertion section 26.

The flexible tube portion 34 has a desired flexibility, and bends by external force. The flexible tube portion 34 is a tubular member extending from a main body portion 38 (to be described later) of the operation section 28.

The operation section 28 includes the main body portion 38, a grasping unit 40, and a universal cord 42. The flexible tube portion 34 extends from a distal end portion of the main body portion 38. The grasping unit 40 is coupled to a proximal end portion of the main body portion 38, and is grasped by the operator who operates the endoscope 12. The universal cord 42 connects the grasping unit 40, on one hand, and the image processing device 14, light source device 18 and light emission/detection device 20, on the other hand.

As illustrated in FIG. 1, the grasping unit 40 is provided with a bend operation unit 36 for operating a plurality of operation wires (not shown) in order to bend the bend portion 32. The bend operation unit 36 includes a left/right bend operation knob 36LR for bend-operating the bend portion 32 in a left-and-right direction, an up/down bend operation knob 36UD for bend-operating the bend portion 32 in an up-and-down direction, and a fixing knob 36C for fixing the position of the bend portion 32 which is bent.

A left/right bend operation driving unit (not shown), which is driven by the left/right bend operation knob 36LR, is connected to the left/right bend operation knob 36LR. In addition, an up/down bend operation driving unit (not shown), which is driven by the up/down bend operation knob 36UD, is connected to the up/down bend operation knob 36UD. The up/down bend operation driving unit and left/right bend operation driving unit are provided, for example, within the grasping unit 40.

The left/right bend operation driving unit is connected to a single left/right operation wire (not shown) which is inserted through the operation section 28, flexible tube portion 34 and bend portion 32. Both ends of this left/right operation wire are connected to the distal end portion of the bend portion 32.

In addition, the up/down bend operation driving unit is connected to a single up/down operation wire (not shown) which is inserted through the operation section 28, flexible tube portion 34 and bend portion 32. The up/down operation wire and left/right operation wire are separate bodies, and can move independently from each other. Both ends of the up/down operation wire are connected to the distal end portion of the bend portion 32.

The left/right bend operation knob 36LR bends the bend portion 32 in the left-and-right direction via the left/right bend operation driving unit and the left/right operation wire. The up/down bend operation knob 36UD bends the bend portion 32 in the up-and-down direction via the up/down bend operation driving unit and the up/down operation wire.

The bend operation unit 36 (left/right bend operation knob 36LR and up/down bend operation knob 36UD), left/right bend operation driving unit, left/right operation wire, up/down bend operation driving unit, and up/down operation wire are a bend operation mechanism which operates the bend portion 32 in order to bend the bend portion 32.

In addition, the endoscope system 10 includes a shape sensor which detects a bend state (bend amount) at a plurality of portions in a predetermined range of the insertion section 26 including the bend portion 32.

Here, although the type of the shape sensor is not limited, a fiber sensor is suitable, which is a bend sensor for detecting a bend from the curvature at a specific location by using an optical fiber. The reasons are (1) the fiber sensor is small in diameter and is easily assembled in the endoscope, and (2) the fiber sensor is less susceptible to the influence of other structural elements and electromagnetic influence. As the shape sensor, a combinational structure of plural strain sensors may be used, aside from the fiber sensor.

In the meantime, if the curvature at a specific location can be found and if the vicinity of the specific location can be regarded as having the same curvature, that is, if the curvature is constant, the bend amount in a range including the specific location can be found. In addition, it can be said that the bend amount is an average curvature in the range including the specific location. Thus, although the curvature and bend amount are different in strict sense, the curvature and bend amount can be regarded as being substantially equivalent if these are limited to the detection values of the shape sensor. Hereinafter, the curvature or bend amount, which is detected by the shape sensor, is referred to as “curvature information”.

As illustrated in FIG. 2, the fiber sensor includes the light emission/detection device 20, an optical fiber 44, a sensing part 46, and a reflection portion 48.

The light emission/detection device 20 includes a light source 20A, a projection lens 20B, an isolator 20C, a reflection mirror 20D, a converging lens 20E, a converging lens 20F, and a bend amount detector 20G.

The light source 20A is, for instance, an LED or the like, and emits light. The projection lens 20B, isolator 20C, reflection mirror 20D and converging lens 20E are disposed on an optical path of light which is emitted from the light source 20A. The converging lens 20F and bend amount detector 20G are disposed on a reflection optical path of the reflection mirror 20D.

The projection lens 20B projects the light which is emitted from the light source 20A.

The isolator 20C passes light from one direction, and blocks light from the other direction. The isolator 20C passes light emitted from the light source 20A, and blocks light from the opposite direction. Thereby, the light, which has passed through the isolator 20C, is converged by the converging lens 20E and is made incident on the optical fiber 44.

The converging lens 20E is disposed between the light source 20A and the optical fiber 44. The converging lens 20E converges the light, which is emitted from the light source 20A, onto the optical fiber 44, so that this light is made incident on the optical fiber 44.

The converging lens 20F converges, on the bend amount detector 200, the light which is reflected by the reflection portion 48, travels back through the optical fiber 44, passes through the converging lens 20E, and is reflected by the reflection mirror 20D.

The reflection portion 48 is disposed in the distal-end rigid portion 30 which is provided at the distal end of the optical fiber 44. The reflection portion 48 reflects light emitted from the optical fiber 44, and makes the light incident on the optical fiber 44 once again.

The reflection mirror 20D passes light from one direction, and reflects light from the other direction. Specifically, the reflection mirror 20D passes the light, which is emitted from the light source 20A and passes through the projection lens 20B and isolator 20C, to the converging lens 20E side. In addition, the reflection mirror 20D reflects return light which is emitted from the optical fiber 44 and passes through the converging lens 20E.

The bend amount detector 20G includes a light receiver such as a light receiving element. The bend amount detector 20G receives incident light and outputs a reception light signal corresponding to the amount of received light, etc. Based on the reception light signal, the bend amount detector 20G outputs a reception light signal corresponding to the magnitude of bend (bend amount) of the bend portion 32.

The optical fiber 44 is inserted from the light emission/detection device 20 to the distal-end rigid portion 30 through the universal cord 42, operation section 28, flexible tube portion 34 and bend portion 32. The optical fiber 44 guides the light, which is emitted from the light source 20A and converged by the converging lens 20E, to the distal-end rigid portion 30 of the insertion section 26 via the operation section 28, as illustrated in FIG. 1. The optical fiber 44 is formed of a line-shaped member.

In addition, at least one above-mentioned sensing part 46 is provided at a position of the optical fiber 44, which corresponds to a predetermined range of the insertion section 26. If the optical fiber 44 is bent in accordance with the bending of the insertion section 26, the sensing part 46 emits the light, which is guided in the optical fiber 44, toward the outside of the optical fiber 44, or absorbs this light, in accordance with the bend state of the optical fiber 44.

The amount of light, which is emitted toward the outside of the optical fiber 44 or is absorbed, corresponds to the bend amount of the optical fiber 44. The sensing part 46 is processed so as to leak the light of the amount corresponding to the bend amount of the optical fiber 44 to the outside of the optical fiber 44, or so as to absorb this light. In other words, the sensing part 46 serves as a unit (optical characteristic changing unit) which changes the optical characteristics of the light guided by the optical fiber 44, for example, the light amount, in accordance with the bend state of the insertion section 26. The sensing part 46 is disposed at a location where a bend is to be detected, or near this location, in at least a predetermined range of the insertion section 26, and is disposed, in particular, at the bend portion 32.

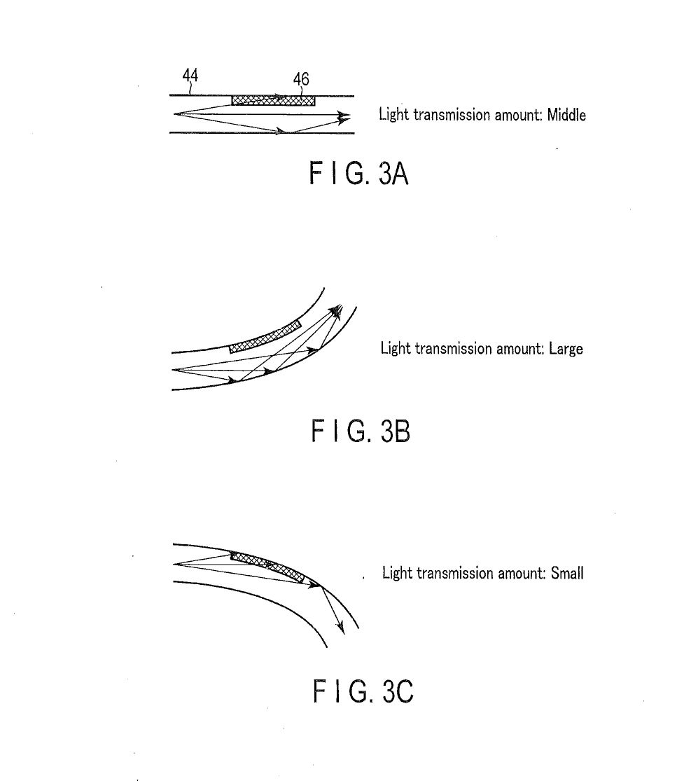

Referring to FIG. 3A to FIG. 3C, the principle of detection of the fiber sensor will further be explained. The fiber sensor is configured such that the optical fiber 44 is provided along the insertion section 26, and the sensing part 46 is provided at a specific location in a predetermined range of the insertion section 26. The fiber sensor is configured to find a bend amount from a curvature of the optical fiber 44.

If the optical fiber 44 changes from a first state (straight state), in which the optical fiber 44 is not bent as illustrated in FIG. 3A, to a state in which the optical fiber 44 is bent, for example, as illustrated in FIG. 3B or FIG. 3C, the amount of light, which is incident on the sensing part 46 provided on the optical fiber 44, varies. FIG. 3B illustrates a second state in which the optical fiber 44 is bent, with the side of the provision of the sensing part 46 being the inside of bending. FIG. 3C illustrates a third state in which the optical fiber 44 is bent, with the side of the provision of the sensing part 46 being the outside of bending. If the first to third states are compared, the light transmission amount by the optical fiber 44 is largest in the second state shown in FIG. 3B, and the light transmission amount by the optical fiber 44 is smallest in the third state shown in FIG. 3C.

If this fiber sensor is used, the shape estimation unit 24 can calculate the bend shape in the predetermined range of the insertion section 26 through which the optical fiber 44 is inserted, based on information from this fiber sensor and prior information. Specifically, the information from the fiber sensor is a bend amount which is indicated by a variation of a reception light signal that is output from the bend amount detector 20G, that is, a variation of optical characteristics, for example, a variation of a light amount, of the light guided in the optical fiber 44 by the sensing part 46 provided on the optical fiber 44. The prior information is a bend direction which is known by the direction in which the sensing part 46 is provided on the optical fiber 44, and a longitudinal-directional position at which the sensing part 46 is provided on the optical fiber 44.

The fiber sensor is a sensor of a light amount variation detection type in which the amount of light traveling in the optical fiber 44 varies due to the bending as described above. The sensor of this type outputs a reception light signal which corresponds to the amount of light varying in accordance with the bending of the insertion section 26 (the bending of the optical fiber 44), that is, the amount of light traveling in the optical fiber 44. Thus, since the detection system can be constituted at low cost, this sensor is suited to mass-produced products.

Aside from the fiber sensor of this light amount variation detection type, there is known a fiber sensor of a so-called FBG type in which a grating is formed on an optical fiber. In this type, although the detection system is complex and the cost tends to increase, the bending can be detected with high precision.

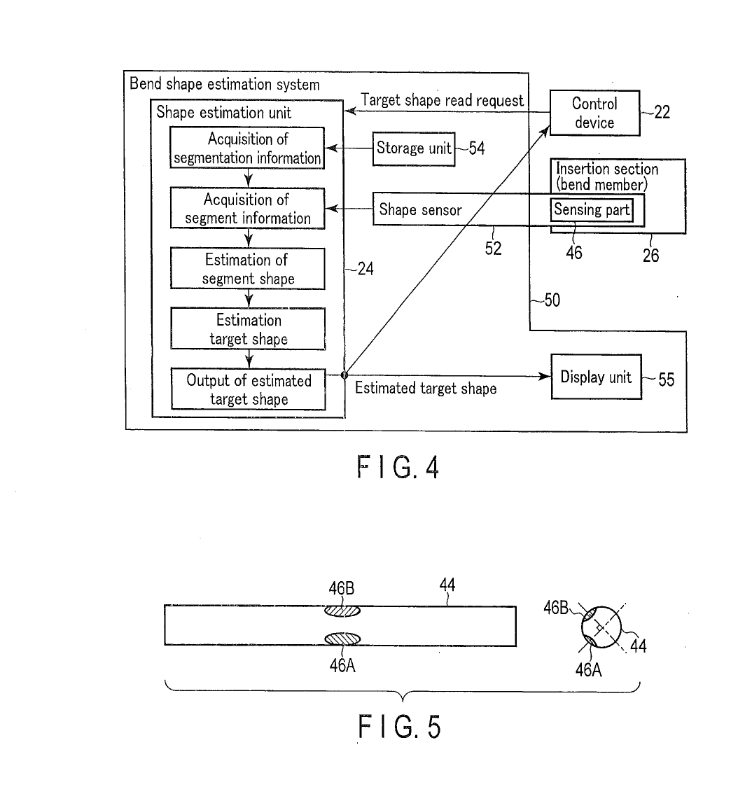

FIG. 4 is a view illustrating the configuration of a bend shape estimation system 50 according to an embodiment of the present invention, which is mounted in the endoscope system 10. This bend shape estimation system 50 includes the above-described shape estimation unit 24, a shape sensor 52 such as the above-described fiber sensor, a storage unit 54, and a display unit (monitor) 55.

The shape sensor 52 is configured such that a plurality of sensing parts 46 are provided in a predetermined range of the insertion section 26 of the endoscope 12. The storage unit 54 stores segmentation information for virtually dividing the optical fiber 44 on which the sensing parts 46 are provided, into a plurality of segments. In the meantime, in the present specification, it is assumed that the segments mean estimation units which virtually neighbor in order in the longitudinal direction in the predetermined range of a rod-like bend member of the target (the insertion section 26 of the endoscope 12 in this case). The estimation range has at least information of a length, a curvature, a shape and a direction for estimating the bend shape of the bend member. The method of division of segments will be described later.

Based on the segmentation information stored in the storage unit 54, the shape estimation unit 24 estimates the bend shape of each of the segments from the curvature information (curvature or bend amount) at the plural sensing part 46, which was detected by the shape sensor 52. In addition, by coupling the estimated bend shapes of the respective segments, the shape estimation unit 24 estimates the bend shape in the predetermined range of the insertion section 26 that is the bend member, on which the plural sensing parts 46 are provided, and displays the estimation result on the display unit 55. In the meantime, the display unit 55 is constituted as a purpose-specific monitor which is different from the display unit 16 of the endoscope system 10. The bend shape in the predetermined range of the insertion section 26 can be presented in juxtaposition with the endoscopic observation image by the display unit 16 of the endoscope system 10.

FIG. 5 illustrates an example in which a fiber sensor capable of detecting a bend amount and a bend direction is used as the shape sensor 54. Sensing parts 46A and 46B are disposed at positions displaced by 90° about the axis of the optical fiber 44.

The reason why the sensing parts are disposed at positions displaced by 90° (i.e. orthogonal positions) about the axis of the optical fiber 44 is that this makes it possible to detect how much the optical fiber 44 is bent in an x-axis direction and a y-axis direction when a coordinate system as shown in FIG. 6 is used. Incidentally, the z-axis is the longitudinal direction of the optical fiber 44, that is, the longitudinal direction of the insertion section 26. An arrangement other than 90°, for example, an arrangement at three points at intervals of 120°, may be adopted. However, in the arrangement at the three points, the arithmetic operations of the bend amount and bend direction become complex, or sensing parts 46 of three or more directions are needed per point. Thus, the two-point arrangement at positions differing by 90°, as illustrated in FIG. 5, is the simplest configuration. In addition, according to this arrangement, the bend characteristics of the optical fiber 44 at the same position in the longitudinal direction can be measured. As a result, when a target (e.g. the insertion section 26 of the endoscope 12), through which the optical fiber 44 is inserted, is divided into segments, as will be described later, two or more different bend detection directions can easily made common with respect to all segments. In addition, such segmentation method can be implemented that sensing parts 46x, 46y of all different directions are disposed at the centers of the segments, and the positions of detection of curvature information (curvature or bend amount) are made uniform in all segments.

Moreover, in each of the segments, one sensing part 46 for detecting the bend of the segment is assigned to one bend direction. Thus, efficient bend detection, with no redundancy, can be performed.

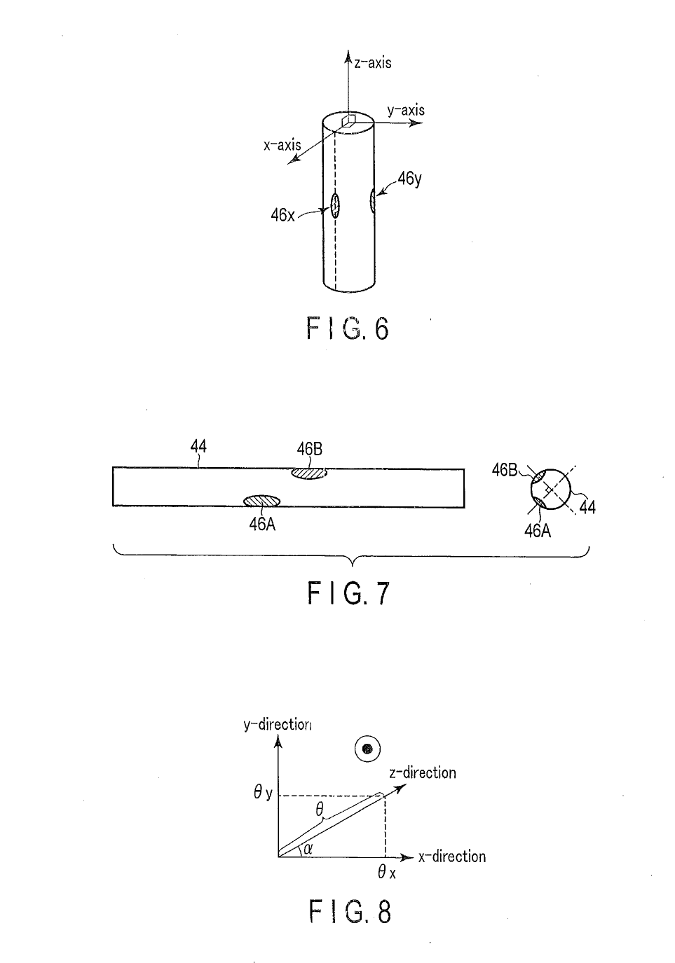

In the meantime, the arrangement method of providing the sensing parts 46 at different positions about the axis of the optical fiber 44 can be variously set, and any arrangement method may be used. For example, it is possible to provide the sensing parts 46 on different optical fibers 44, or to slightly displace, as illustrated in FIG. 7, the positions of the sensing parts 46A and 46B in the longitudinal direction of the optical fiber 44.

By slightly displacing, as illustrated in FIG. 7, the positions of the two sensing parts 46A and 46B in the longitudinal direction of the optical fiber 44, it becomes possible to avoid overlapping of the two sensing parts 46A and 46B with large widths. Further, it is possible to avoid the deficiency in structural strength due to the provision of the two sensing parts 46A and 46B, a variation in bend characteristics of the optical fiber 44, and degradation in reliability. Besides, the bend characteristics at substantially the same position in the longitudinal direction of the optical fiber 44 can be measured. As a result, when the target (e.g. the insertion section 26 of the endoscope 12), through which the optical fiber 44 is inserted, is divided into segments as will be described later, the two or more different bend detection directions can easily be made common with respect to all segments. In addition, such segmentation method can be implemented that the sensing parts 46 of all different directions are disposed substantially at the centers of the segments, and the positions of detection of curvature information (curvature or bend amount) are made uniform in all segments.

FIG. 8 illustrates an example of detection of a bend amount and a bend direction. In this example, the detection results of bend amounts in the x-axis direction and y-axis direction are used. Thereby, when the bend amounts in the x-direction and y-direction are θx, θy, the bend amount is θ, and the angle of the bend direction to the x-axis is α, the following is given:

θ=sqrt(θx²+θy²), and

θ cos α=θx, θ sin α=θy (equation 1).

Accordingly, the shape estimation unit 24 detects the bend amount and bend direction such that “a bend by θ occurs in a bend direction which is rotated by a from the x-axis.”

Incidentally, the method of detection of the bend amount and direction does not need to be always restricted to this method.

In addition, the curvature of bend may be used in place of the bend amount. An example of calculation of the bend direction and curvature at this time is shown.

When
the curvatures in the x-direction and y-direction are 1/Rx and 1/Ry,

1/R=sqrt{(1/Rx)²(1/Ry)²}, and

1/R·cos α=1/Rx, 1/R·sin α=1/Ry (equation 2),

it is assumed that “a bend by curvature 1/R occurs in a direction which is rotated by α from the x axis.”

Accordingly, by using as the shape sensor 52 the fiber sensor in which the sensing parts 46A and 46B are disposed as illustrated in FIG. 5 or FIG. 7, the shape sensor 52 can be obtained which detects the bend shape in a desired range of the insertion section 26 of the endoscope 12.

The bend characteristics at the sensing parts 46, which were detected by the shape sensor 52 such as the fiber sensor, that is, the curvature information (curvature or bend amount) at the sensing parts 46, are input to the shape estimation unit 24. Based on the detected bend characteristics, the shape estimation unit 24 calculates the partial bend shape of the target, that is, the bend shape of segments.

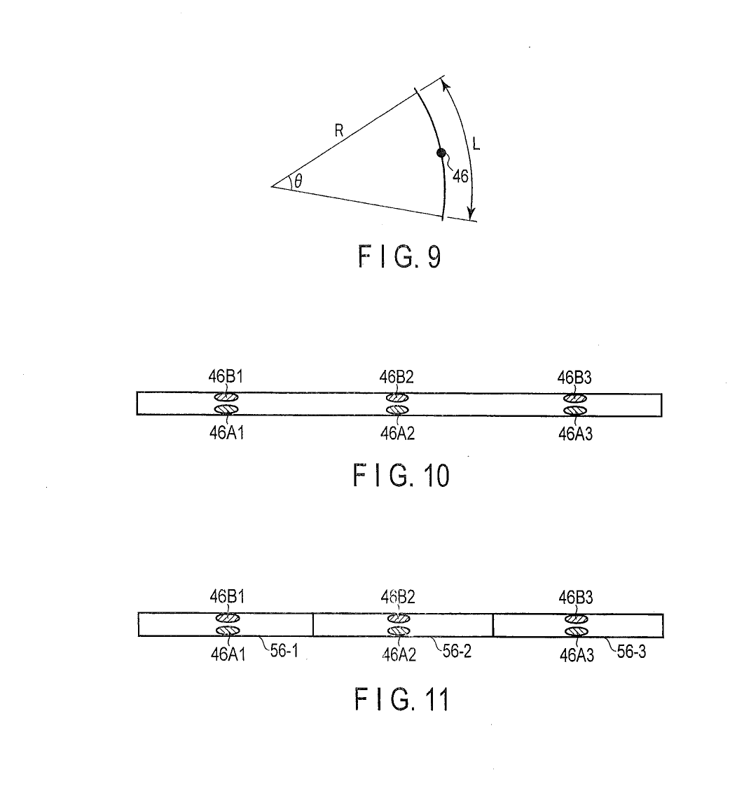

FIG. 9 illustrates an example of a concrete bend shape which is detected by the shape sensor 52.

If the detected curvature at the sensing part 46 is 1/R and the length of the detection range (segment) is L, a relationship “L=Rθ” exists between the radius R, length L and bend angle θ (the unit of θ is radian), when it is assumed that the target is bent in an arcuate shape. Accordingly, the bend angle θ is given as “θ=L/R”.

In this manner, by estimating the shape of the target as an arcuate shape, the shape can easily be estimated like the above equation 1 or equation 2. In the shape estimation by the connection of segments, which will be described later, when a numerical arithmetic process is performed, the position and direction of the other end relative to one end can be calculated by only the combination of processes of movement and rotation, and the processing is easy.

In the example of FIG. 9, the shape estimation is performed by regarding the bend shape of the vicinity of the sensing part 46 as an arcuate shape, but other shape estimations may be performed. In the case of an arc, the direction of bend and curvature are constant regardless of locations. However, for example, such a shape is possible that at least either the direction of bend or curvature varies depending on locations. In addition, it is possible to regard the shape as a straight line (line segment), and to estimate the angle or direction between line segments at a connection part to the shape of the vicinity of neighbor another sensing part 46. Besides, it is possible to use a method in which a reference table is used in order to estimate the bend shape from the detection result at the sensing part 46.

Next, an example is described in which the bend shape estimation in FIG. 5 is further developed, and bend shape detection is performed in an optical fiber sensor in which sensing part 46 are provided at a plurality of locations.

<Description of Segments>

FIG. 10 illustrates an example of using a fiber sensor which can perform bend detection at a plurality of locations.

Six sensing parts 46A1, 46B1, 46A2, 46B2, 46A3 and 46B3, in total, are disposed on one optical fiber 44, such that two sensing parts are disposed at two locations displaced by 90° about the axis of the optical fiber 44, and this pair is disposed at three locations in the longitudinal direction. In the meantime, even if the number of sensing parts 46 is equal, a plurality of optical fibers 44 may be used, or the positions of the sensing parts 46 may be varied. In such manners, the method of arrangement of sensing parts 46 can variously be set, and any method of arrangement may be used.

Incidentally, it is assumed that the bend direction about the axis of the optical fiber 44 is defined in the state in which the longitudinal-axis direction of the optical fiber is straight, as illustrated in FIG. 10. The same applies to the bend direction of the target.

Here, consideration is given to general cases including shape sensors other than the fiber sensor. The longitudinal direction, which is a straight-line direction in the straight state of the target to be estimated the bend shape, as illustrated in FIG. 6, is set as a z-axis. A direction perpendicular to the z-axis is set as an x-axis, and a direction perpendicular to the z-axis and x-axis is set as a y-axis. As regards the coordinate system (xyz axes) at an arbitrary point on the target, the longitudinal direction is always set as the z-axis even at a time of bending, and the x-axis and y-axis are affected by only the influence of rotation by bending. Specifically, it is assumed that the sensing part 46x, which is located in the x-axis direction in the straight state, is still in the x-axis direction even if the target is bent. For example, if the x-axis direction of the target or optical fiber 44 is marked, the marked direction is the x-axis direction even if the target is bent. It is assumed that the bend direction at a time of bending is judged from the x-axis direction and y-axis direction in the straight state or at the time of bending of the target. In particular, when the bend directions are compared between segments, this method is adopted.

In addition, the directions of the x-axis and y-axis may be arbitrarily set on a point-by-point basis on the target. However, convenience is high if the directions of the x-axis and y-axis are uniform at all points on the target when the target is in the straight state, and, in this example, too, it is assumed that the directions of the x-axis and y-axis are uniform at all points on the target.

In order to calculate the bend direction, the sensing parts 46x and 46y for detecting the curvature information (curvature and bend amount) in two or more different directions, which are perpendicular to the z-axis, need to correspond in all segments. In this example, the sensing parts 46x and 46y are disposed in directions displaced by 90°.

In order to apply the example of bend detection at one location, as illustrated in FIG. 5 or FIG. 7, and FIG. 6 and FIG. 8 and also in FIG. 9, to the example of FIG. 10, an example is described in which the target (e.g. the insertion section 26 of the endoscope 12), through which the optical fiber 44 is inserted, is divided into three segments 56 (56-1, 56-2, 56-3), as illustrated in FIG. 11.

In FIG. 11, the segments 56-1, 56-2 and 56-3 include, respectively, sensing parts 46A1 and 46B1, sensing parts 46A2 and 46B2, and sensing parts 46A3 and 46B3 in directions differing by 90°. Based on the curvatures of the sensing parts 46A1, 46B1, 46A2, 46B2, 46A3 and 46B3, the bend amounts and bend shapes of the respective segments 56-1, 56-2 and 56-3 can be calculated as illustrated in FIG. 6, FIG. 8 and FIG. 9.

By connecting the calculated bend amounts and bend shapes of the respective segments 56-1, 56-2 and 56-3, the bend shape in the entire detection effective region, which is the above-described predetermined range, can be estimated.

The conditions at a time of the connection are the following three:
1) The segments are continuously connected at connection parts between the segments,
2) The directions (tangent directions) of the respective segment end portions coincide at the connection parts between the segments, and
3) The segments are connected at the connection parts between the segments, with no twist or rotation of the segments 56.

As regards the above conditions, it is assumed that, in FIG. 6, not only the z-axis direction, but also the x-axis direction and y-axis direction in condition (2) are made to coincide at the connected end portions, so that the x-axis direction and y-axis direction about the z-axis may not be displaced at the time of connecting the segments 56.

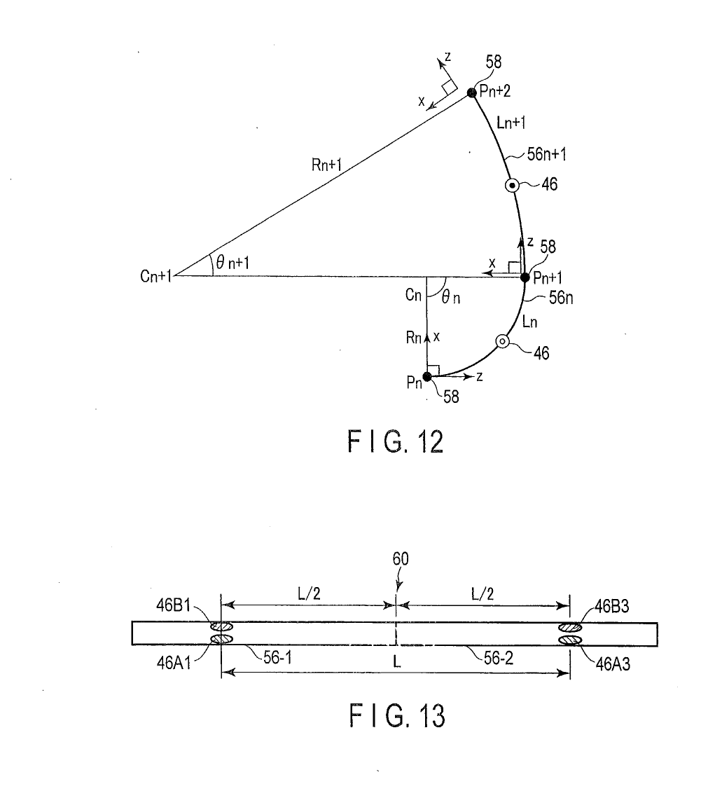

FIG. 12 illustrates an example of the connection of two segments 56 (an n-th segment 56n and an (n+1)th segment 56n+1) according to this connection method. For the purpose of simplicity, an example of a bend in the same plane is illustrated. At three connection points 58, the x-axis and z-axis of the coordinate system xyz are shown. In this example, at all connection points 58, the direction of y-axis is an upward direction on the sheet of the drawing.

As regards the n-th segment 56n, the positions of connection points 58 at both ends are Pn and Pn+1, the length is Ln, the radius of curvature is Rn, the bend amount (bend angle) is θn, and the center of curvature is Cn. At this time, at the position Pn and position Pn+1, the direction toward the position Cn is perpendicular to the tangent of the segment 56n.

Similarly, as regards the (n+1)th segment 56n+1, the positions of connection points 58 at both ends are Pn+1 and Pn+2, the length is Ln+1, the radius of curvature is Rn+1, the bend amount (bend angle) is θn+1, and the center of curvature is Cn+1. At this time, at the position Pn+1 and position Pn+2, the direction toward the position Cn+1 is perpendicular to the tangent of the segment 56n+1.

Of the three connection conditions, from the condition (1), the position Pn+1 is common to the two segments 56n and 56n+1. In addition, from the condition (2), the tangent directions of the two segments 56n and 56n+1 coincide at the position Pn+1. Further, from the condition (3), the segments are connected with no twist (with no rotational displacement about the tangent direction), that is, with the directions about the tangent direction being coincident.

In the meantime, when the bend directions of the two segments 56n and 56n+1 are different, the bend shape becomes a three-dimensional structure, and the positions Pn, Pn+1 and Pn+2 do not fall in the same plane. In addition, the position Pn+1, position Cn and position Cn+1 are arranged so as not to be on the same straight line.

In this manner, the sensor 52, such as the fiber sensor, which can detect the curvatures or bend angles at plural locations of itself, is used, at least a part thereof is divided into segments 56, the bend shape of each segment 56 is calculated as an arcuate shape, and the respective segments 56 are connected to calculate the bend shape of at least a part of the shape sensor 52. Thereby, the bend shape of the shape sensor 52 can easily be found. As a result, if a position, which becomes a reference, is provided in the range in which the bend shape is understood, a position or a distance from this reference can be found.

In particular, with respect to each segment 56, by using the curvature information (curvature or bend amount) in two different directions such as the x-axis and y-axis in FIG. 6, it becomes possible to detect not only the bend amount of each segment 56, but also the direction of bend. Thus, it is also possible to detect a three-dimensional bend shape. In this case, when the two different directions are orthogonal directions, the bend amount of each segment 56 can be calculated by simple mathematical expressions like the above-described equations (1) and (2). In particular, as illustrated in FIG. 11, when the respective segments 56-1, 56-2 and 56-3 include the sensing parts 46A1, 46B1, 46A2, 46B2, 46A3 and 46B3 which are provided in the two orthogonal directions, the sensor which directly measures the curvature information (curvature or bend amount) of each segment 56-1, 56-2, 56-3 is assembled, and the exact shape detection can be performed without estimation of the bend amount, etc.

With respect to the predetermined range of this segmented bend member (insertion section 26), the shape estimation unit 24 shown in FIG. 1 also executes the connection of the estimated bend shapes of the respective segments 56 and the estimation of the bend shape in the predetermined range of the bend member.

<Explanation of Segmentation>

In the above, the description has been given of the method of estimating the bend direction and curvature of each of the segments 56 into which the bend member was segmented. When the shape sensor 52 is assembled in the bend member of the tubular insertion system such as the insertion section 26 of the actual endoscope 12, it is necessary to determine the method of segmentation in accordance with the manner of bending of the bend member.

The object of segmentation is to clarify the unit of calculation of the bend shape (curvature and bend direction).

<Segment Boundary at Midpoint>

FIG. 13 illustrates a method of determining a boundary between segments 56.

It is assumed that there are two neighboring segments 56-1 and 56-2, and there is a distance L between the sensing part 46A1, 46B1 and the sensing part 46A2, 46B2. At this time, a segment boundary 60, which is a boundary between the segments 56-1 and 56-2, is set at a midpoint which is at a distance of L/2 from the sensing part 46A1, 46B1 and from the sensing part 46A2, 46B2.

In addition, as illustrated in FIG. 10, as regards the part at which the endmost sensing part 46 is present, the boundary of the segment 56 may be set up to the end portion of the sensor. Further, for example, in the fiber sensor, if the optical fiber with no sensing part 46 extends over a great length, the segment boundary 60 may be provided at the same length as the length from the sensing part 46 to the segment boundary 60 on the opposite side. A range with no sensing part 46 beyond this segment boundary 60 is outside the shape detection range of the shape sensor 52.

In this manner, the midpoint between the sensing parts 46 of the neighboring segments 56 is set as the segment boundary 60. Thereby, the method of determining the segment boundary 60 is very simple, needless to say, not only in the case in which the bend characteristics of the bend member that is the measurement target of the bend shape are substantially constant regardless of locations in the longitudinal direction, but also in the case in which the bend characteristics vary depending on the condition of use, and in the case in which the bend characteristics, per se, are unclear. In addition, on the assumption that each of the segments 56 is similarly bendable, the bend shape of each segment 56 can be measured with substantially the same detection sensitivity and detection range.

<Large Variation (1) of Bend Characteristics at Segment Boundary: Active and Passive Bends>

Here, a description is given of the method of determining the boundary of segments 56 in a part where the bend characteristics sharply change at the segment boundary 60.

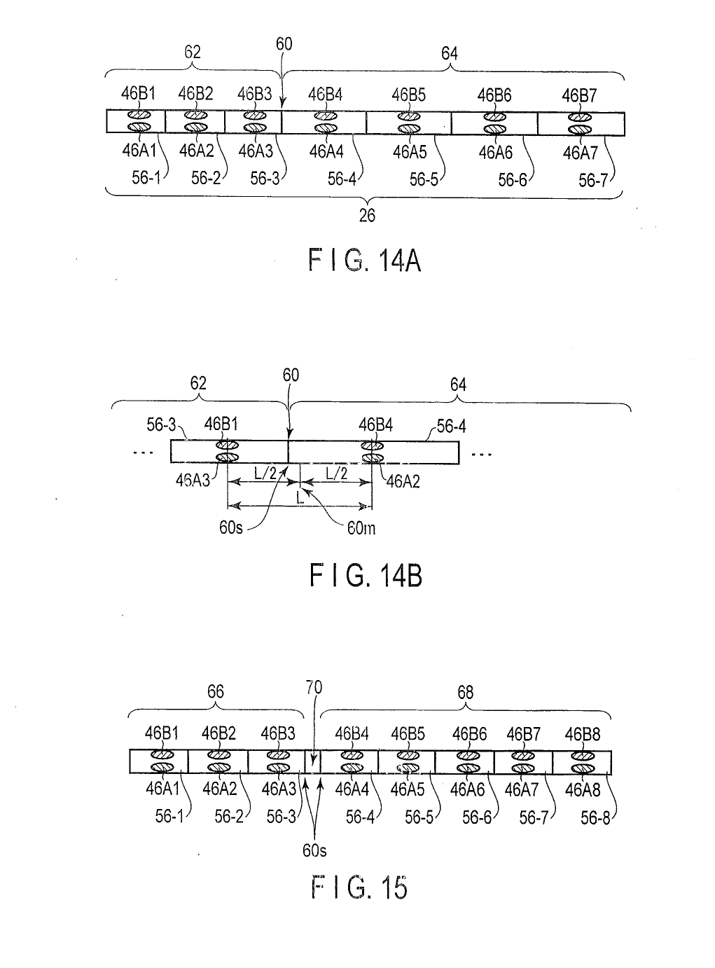

FIG. 14A illustrates an image of the insertion section 26 of the endoscope 12. The left side in the Figure is a distal end side of the insertion section 26 and is an active bend portion 62 such as the bend portion 32, which is operable by the bend operation unit 36. The right side is a passive bend portion 64 such as the flexible tube portion 34, which is bent by external force received from the operator or from a lumen. The active bend portion 62 and passive bend portion 64 have different characteristics. The active bend portion 62 is particularly easily bendable at least in one direction.

A part where the bend characteristics are greatly different is set as a second boundary 60s. When the second segment boundary 60s is located at a position different from a segment boundary 60m which is determined by the midpoint between the sensing parts 46 of the neighboring segments as illustrated in FIG. 13, the second segment boundary 60s is preferentially selected as illustrated in FIG. 14B, and is set as the actual segment boundary 60.

In addition, in the active bend portion 62, segments 56-1, 56-2 and 56-3, which are fine and substantially equal in width, are arranged in the longitudinal direction. In the passive bend portion 62, segments 56-4, 56-5, 56-6 and 56-7, which are long and substantially equal in width, are arranged in the longitudinal direction.

When bend characteristics are sharply different on both sides of the connection portion at which such portions with different bend characteristics are connected, if the connection portion exists in one segment, the estimation of the bend shape becomes complex. Conversely, if the connection portion that is the second segment boundary 60s is set as the segment boundary 60, the segments 56-1, 56-2 and 56-3 and segments 56-4, 56-5, 56-6 and 56-7 with substantially fixed bend characteristics can be arranged on both sides of the connection portion, the shape estimation is easy, and high-precision shape estimation can be expected. In particular, such effects can be expected by the combination with the application of the segment boundary 60, by which the midpoint between the sensing parts 46 of the neighboring segments 56 is set as a segment boundary 60m, as shown in FIG. 13.

Besides, the bend member including the active bend portion 62 and passive bend portion 64 is particularly widely used, for example, in the insertion section 26 of the endoscope 12. By adopting such a segmentation method, the shape of the insertion section 26 can be easily estimated with high precision, and thus an improvement in insertion and operability can be expected.

Moreover, by assembling and using, as the shape sensor 52, the fiber sensor in which the diameter of the optical fiber 44 is about φ0.1˜0.5 (mm), as illustrated in FIG. 1, the shape estimation can be performed without substantially increasing the diameter of the insertion section 26 that is the bending member, without the influence of disturbance, or without an external antenna, etc. As a result, the shape estimation in the predetermined range of the bend member can be performed without changing the functions and specifications of the endoscope 12.

<Large Variation (2) of Bend Characteristics at Segment Boundary: Connection Portion of Rigid Body>

A description is given of another example of the method of determining the boundary of segments in a part where the bend characteristics sharply change at the segment boundary 60.

In FIG. 15, the left side is a distal-end bend portion 66, and the right side is a proximal bend portion 68. A rigid portion 70 exists at a connection portion between the distal-end bend portion 66 and proximal bend portion 68. In this structure, in many cases, the shapes of lumens for insertion are different between the distal-end side and proximal side, with the rigid portion 70 being the boundary, and the purpose of use is different between the distal-end side and the proximal side. For example, the proximal side is disposed in a path for reaching a target internal organ. When the distal side is in the path, the switching of the direction of insertion is executed, and when the distal side is at the part of the target internal organ, more specific selection of paths and treatment such as observation or therapeutic treatment are performed. In this manner, the purpose of use and the disposition of contents of lumens are different between the front side and rear side of the connection portion that is the rigid portion. Even if the bend characteristics of the proximal and distal sides are equal, differences occur in shapes in many cases.

Thus, both ends of the rigid portion 70, which is the part at which the bend characteristics sharply differ, are set as second segment boundaries 60s. In the meantime, if the length of the rigid portion 70 is very small, only the center of the rigid portion 70 may be set as a segment boundary 60. Conversely, if the length of the rigid portion 70 is not very small, the rigid portion 70 may be called “rigid segment” as a segment 56 that does not change its shape. When the second segment boundary 60s is present at a position different from the segment boundary 60m that is determined by the midpoint between the sensing parts 46 of the neighboring segments, as illustrated in FIG. 13, the second segment boundary 60s is preferentially selected and is set as the actual segment boundary 60.

In this manner, when different shapes are taken between the front and rear sides of the connection portion that is the rigid portion, the connection portion is set as the segment boundary 60. Thereby, the segments 56-1, 56-2 and 56-3 and segments 56-4, 56-5, 56-6, 56-7 and 56-8 with substantially fixed bend characteristics can be arranged on both sides of the connection portion, and easy and high-precision shape estimation can be expected. In addition, on the front and rear sides of the connection portion, such effects can be expected by the combination with the application of the segment boundary 60, by which the midpoint between the sensing parts 46 of the neighboring segments is set as a segment boundary 60m, as shown in FIG. 13.

<Determination of Segment Boundary by Bend Amount>

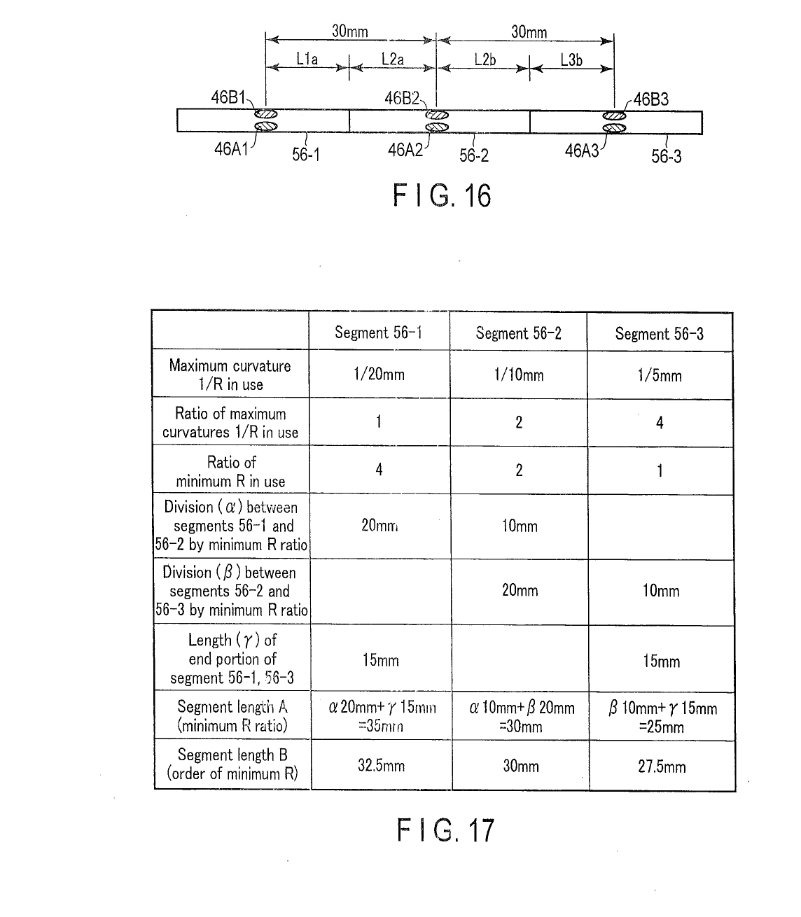

In FIG. 16, when a portion with a fixed length, on which sensing parts 46 are arranged at regular intervals, is divided into three segments 56-1, 56-2 and 56-3, the precision of detection of the bend shape is determined by how to set the segment lengths of the segments 56-1, 56-2 and 56-3.

For example, the following three characteristics can be mentioned as concrete indices of segmentation:
(1) Maximum curvature 1/R in use (an inverse number of a minimum bend radius R in use),
(2) Maximum curvature 1/R to be detected (an inverse number of a minimum bend radius R to be detected), and
(3) Flexural rigidity EI.

The values of these indices have, in some cases, sharply different distributions, depending on parts where the segments 56 are provided. When the boundary between the segments 56 of the bend member that is the target with a distribution of different bend characteristics, for example, the insertion section 26 of the endoscope 12, is to be determined, it is desirable to determine the segment length in accordance with the indices of the bend member.

In any case, these indices (1) to (3) or the inverse numbers of the indices (the inverse numbers of (1) and (2), and the value of index (3) as such) are used, and the segment boundaries 60 are set in an order of the ratios of values of these indices, or in an order of the values of these indices.

<Part 1: Determination of Segment Length Based on Minimum Bend Radius R in Use>

As regards the above index (1), “Maximum curvature 1/R in use (an inverse number of a minimum bend radius R in use)”, if the curvature 1/R in specifications or in an actual bend range is large, that is, if the radius R is small, it is necessary to set the segment length at a small value. By setting the segment length in proportion to the inverse number of the magnitude of the value of the index (1), segmentation conforming to the index can be implemented.

For example, as shown in FIG. 17, when the maximum curvatures 1/R in use of the segments 56-1, 56-2 and 56-3 are 1/20 mm, 1/10 mm and 1/5 mm, the ratio of the inverse numbers of the maximum curvatures 1/R in use (the minimum R in use) is 4:2:1. In FIG. 16, if the total segment length is 90 mm and the interval between sensing parts 46 is 30 mm, L1a:L2a=4:2, and L2b:L3b=2:1. Since L1a+L2a=L2b+L3b=30 mm, L1a=20 mm, L2a=10 mm, L2b=20 mm, and L3b=10 mm. This result is shown in the field of segment length A in FIG. 17.

In addition, by setting the segment lengths in the order of inverse numbers of the magnitudes of the values of the index (1), segmentation conforming to the respective indices to some degree can be implemented. In the example of FIG. 17, a combination of values as shown in the row of segment length B can be assigned as intermediate values between the simple uniform division (trisection) and the ratio of inverse numbers of the index (1).

<Part 2: Determination of Segment Length Based on Minimum Bend Radius R to be Detected>

Similarly, as regards the above index (2), “Maximum curvature 1/R to be detected (an inverse number of a minimum bend radius R to be detected)”, if the curvature 1/R in the range to be detected is large, that is, if the radius R is small, it is necessary to set the segment length at a small value.

Like the case of the above index (1), by actually setting the segment length in proportion to the magnitude of the inverse number of the value of the index (2), segmentation conforming to the respective indices can be implemented. In addition, by actually setting the segment lengths in the order of the magnitudes of the inverse numbers of the values of the indices (1), (2) and (3), segmentation conforming to the respective indices to some degree can be implemented.

<Part 3: Determination of Segment Length Based on Flexural Rigidity EI>

As regards the flexural rigidity EI that is the index of (3), E and I are first explained. E is a Young's modulus that is an index of difficulty in bending, which is determined by physical properties of material. I is a geometrical moment of inertia that is an index of difficulty in deformation of a body relative to a bending moment, which is determined by the cross-sectional shape. A product EI of E and I becomes an index of difficulty in bending by the member and cross-sectional shape. If EI is small, the ease in bending increases, and it is thus necessary to set the segment length at a small value.

Like the cases of the above indices of (1) and (2), by actually setting the segment lengths in proportion to the magnitude of the value of the index (3), segmentation conforming to the respective indices can be implemented.

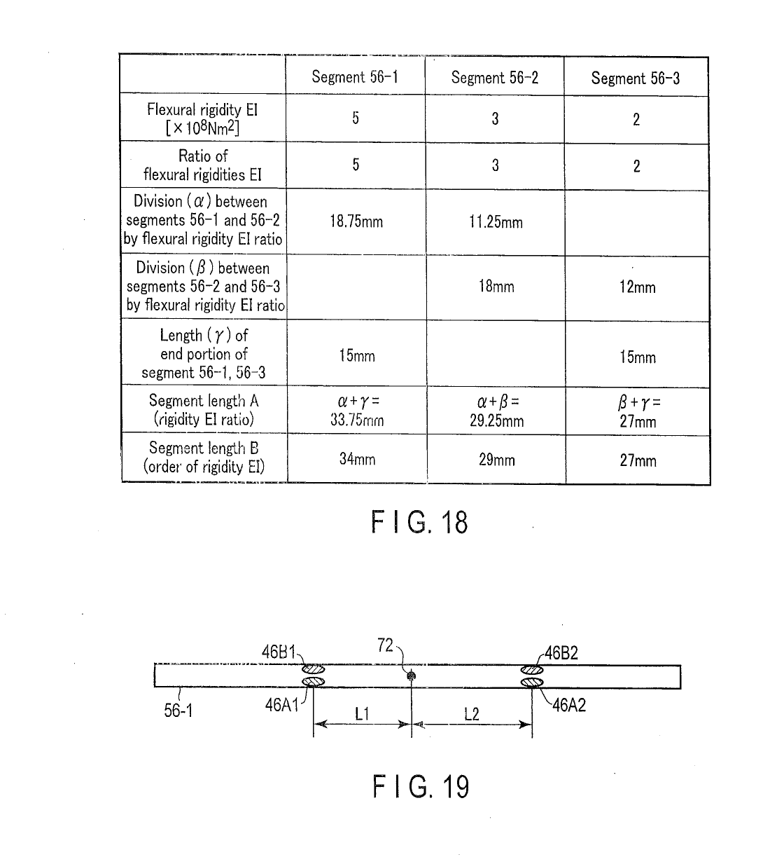

For example, as illustrated in FIG. 18, when the flexural rigidities EI in use of the segments 56-1, 56-2 and 56-3 are 5, 3, and 2 [×10⁸ Nm²], the ratio of rigidities becomes 5:3:2. At this time, in FIG. 16, if the total segment length is 90 mm and the interval between sensing parts 46 is 30 mm, L1a:L2a=5:3, and L2b:L3b=3:2. Since L1a+L2a=L2b+L3b=30 mm, L1a=18.75 mm, L2a=11.25 mm, L2b=18 mm, and L3b=12 mm. This result is shown in the field of segment length A in FIG. 18.

In addition, as shown in the field of the segment length B in FIG. 18, by actually setting the segment lengths in the order of the magnitudes of the values of the indices (3) of the segments 56-1, 56-2 and 56-3, segmentation conforming to the respective indices to some degree can be implemented.

In this manner, in the tubular system such as the endoscope 10 in which the shape sensor 52 such as the fiber sensor capable of detecting the curvatures or bend angles at plural locations of itself is mounted on the bend member, at least a part of the tubular system is divided into segments 56, the bend shape of each segment 56 is found as an arcuate shape, and the respective segments 56 are connected to calculate the bend shape of at least a part of the shape sensor 52. Thereby, the bend shape in a predetermined range of the bend member of the tubular system, for example, the insertion section 26 of the endoscope system 10, can be easily found. In particular, as regards the fiber sensor, the diameter is small and wiring or the like is needless. Thus, the fiber sensor can suitably be mounted on the tubular system.

In addition, as regards the segmentation, in order to optimize the number of segments 56 or sensing parts 46 of the shape sensor 52, it is desirable to determine the segment length in accordance with the ease in bending or the bend amount of the bend member, for example, the insertion section 26 of the endoscope 12. As concrete indices, the three indices, (1) the maximum curvature 1/R in use, (2) the maximum curvature 1/R to be detected and (3) the flexural rigidity EI, were mentioned.

As regards (1) the maximum curvature 1/R in use and (2) the maximum curvature 1/R to be detected, the segment boundary 60 is set based on these indices, such that the interval of the sensing parts 46 of the segments 56 corresponds to such segment lengths that the bend amounts become equal or close to each other. Thereby, the detection sensitivity of the shape sensor 52 can be improved. If the segment boundary 60 is set in accordance with the indices, the optimal segment length is obtained if there is no other factor affecting the segment length. In addition, when the detection sensitivity is determined in combination with other factors, more suitable bend detection is enabled by setting the bend amounts of the respective segments 56 at close values.

In addition, as regards (3) flexural rigidity EI, the segment length is set based on this index such that the bend amounts of the respective segments 56 become equal or close to each other. Thereby, the segment length relative to the ease in bending of the bend member, for example, the insertion section 26 of the endoscope 12, can be optimized.

If the bend amounts of the segments 56 at a time when the same bending moment is applied are made to coincide, the optimal segment length is obtained when there is no other factor affecting the segment length. In addition, when the detection sensitivity is determined in combination with other factors, more suitable bend detection is enabled by setting the bend amounts of the respective segments 56 at close values.

<Number of Sensing Parts in Segment>

When not only the bend amount but also the bend direction is to be detected, sensing parts, which are disposed in two or more different directions, are needed. For example, as illustrated in FIG. 6, if the sensing parts 46x and 46y are disposed along the x-axis and y-axis in directions which are displaced by 90° and are perpendicular to the longitudinal direction of the bend mechanism, the number of necessary sensing parts 46 can be minimized. Alternatively, sensing parts 46 may be disposed in three directions at intervals of 120°, or in four directions at intervals of 90°. In this case, the bend direction and curvature information (curvature or bend amount) of each segment 56 may be found based on the curvature detection values of the three or four sensing parts 46. By the increase in number of sensing parts 46 for detecting the bend direction and bend amount of segments, the precision and stability of detection can be improved.

Furthermore, when a plurality of sensing parts 46 for detecting the same bend direction are disposed in one segment, the detection value at one sensing part 46 may be used, or determination may be made by performing such weighting as to be inversely proportional to the distances from the position representative of the bend shape of the segment (the center in the longitudinal direction when the position is not specifically designated) to the respective sensing parts 46.

A concrete method of weighting is described with reference to FIG. 19.

In a segment 56-1, there are four sensing parts 46 in total, namely sensing parts 46A1 and 46A2 for the x-axis direction, and sensing parts 46B1 and 46B2 for the y-axis direction. Here, the distances from the sensing parts 46A1 and 46B1 and the sensing parts 46A2 and 46B2 to a point (black circle in the FIG. 72 representing the segment 56-1 are set as L1 and L2, the detection values at the sensing parts 46A1, 46A2, 46B1 and 46B2 are set as CA1, CA2, CB1 and CB2, the detection value in the x-axis direction is set as CA, and the detection value in the y-axis direction is set as CB. At this time, by performing weighting as shown below, the detection values, which are assumed, are calculated:

CA=L2/(L1+L2)·CA1+L1/(L1+L2)·CA2, and

CB=L2/(L1+L2)·CB1+L1/(L1+L2)·CB2.

<Segment Arrangement Outside Detection Effective Range>

As described above, in the detection effective area that is the predetermined range of the bend member (e.g. the insertion section 26 of the endoscope 12) which is the detection target, the segments are arranged so as to neighbor each other. In this effective detection area that is the predetermined range, for example, in the case of the insertion section 26 of the endoscope 12, the entirety of the bend portion 32 is indispensable, but the flexible tube portion 34 may include only a portion of an arbitrary length from the distal end side thereof, which is continuous with the bend portion 32. The reason for this is that the entirety of the flexible tube portion 34 is not inserted in a lumen of a subject, and there is little need to recognize the bend shape of that part of the flexible tube portion 34, which is inserted in the lumen, except for the vicinity of the bend portion 32. Accordingly, there is no need to provide the sensing part 46 outside this effective detection area that is the predetermined range.

However, even on the outside of the effective detection area, sensing parts 46 may be provided more sparsely than in the predetermined range, so that a rough bend shape of the bend portion in the lumen can be viewed. In this case, the segment 56 is not always necessary. Thus, on the outside of the effective detection area, there may be some distance from other segments 56, or the segment length in the longitudinal direction of the bend portion may be large.

<Bend Shape Estimation Method of Bend Member>

Next, a description is given of a method of estimating the bend shape in the predetermined range of the bend member (e.g. the insertion section 26 of the endoscope 12) that is the detection target, in the bend shape estimation system 50 as illustrated in FIG. 4.

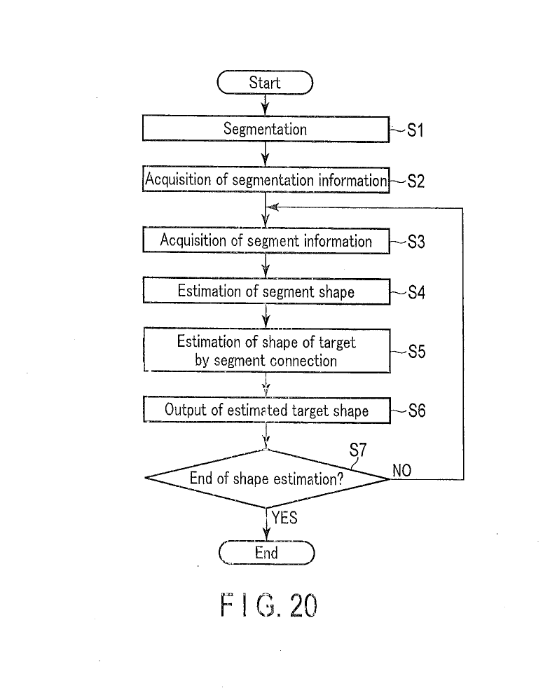

The estimation method includes the following seventh steps, as illustrated in FIG. 20.

To start with, segmentation is implemented (step S1). This step is a step in which a predetermined range with flexibility of the insertion section 26 of the endoscope 12 that is the bend member, which is inserted in a lumen of a subject and performs a predetermined work, is segmented into a plurality of segments 56 such that at least one of a plurality of sensing parts, which are disposed along the longitudinal direction of the bend member, is included in each of the respective segments 56.

For example, this segmentation is implemented at a time of design for attaching the shape sensor 52 to the insertion section 26 that is the bend member, or implemented while checking the characteristics of the insertion section 26 that is the bend member, in which the shape sensor 52 was actually assembled. In the case of a product, segmentation is implemented before factory shipment.

Since the segmentation can be executed offline, the segmentation may be performed by a designer, or may be executed by a computer on the system or not on the system.

When the segmentation as illustrated in FIG. 11 is implemented on the arrangement of sensing parts 46 as illustrated in FIG. 10, the midpoint between the sensing parts 46 allocated to the neighboring segments 56 is set as the segment boundary 60, as illustrated in FIG. 13. However, as illustrated in FIG. 14A or FIG. 15, at a part where bend characteristics sharply change, this part of the change is preferentially set as the segment boundary 60.

In addition, as illustrated in FIG. 16 to FIG. 18, when bend characteristics are different from portion to portion, the segment boundary 60 between the sensing parts 46 allocated to the neighboring segments 56 is determined in accordance with the ease in bending (the bend amount relative to a predetermined bending moment), the distribution of maximum curvature, etc.

The information necessary for shape estimation, other than curvature information, which is obtained by the segmentation, is stored in the storage unit 54 as segmentation information. The segmentation information includes the disposition of each segment 56, the length of each segment 56, and so on.

Thereafter, the shape estimation unit 24 acquires the segmentation information from the storage unit 54 (step S2). This step is a step of acquiring the information necessary for shape estimation, other than curvature information, such as the disposition and length of each segment 56. This step is executed, for example, when the process by the bend shape estimation system 50 is first executed after power-on. Alternatively, when this bend shape estimation system 50 was applied to the endoscope system 10 that is the tubular insertion system, this step is executed in response to a target shape read request from the control device 22 of the endoscope system 10.

Next, the shape estimation unit 24 acquires the segment information which includes the curvature information (curvature or bend amount) detected by the sensing parts 46 of the shape sensor 52 (step S3). Specifically, the segment information includes first curvature information that is curvature components (1/Rx, 1/Ry) or curvature amount components (θx, θy) with respect to predetermined bend directions (x-direction, y-direction) as illustrated in FIG. 8.

If the shape in a static state is estimated, one-time segment information acquisition is sufficient. However, in order to measure the shape of the bend member which is changing with time, the acquisition and the shape estimation, which is shown below, need to be repeated.

Next, the shape estimation unit 24 executes shape estimation of each segment 56 (step S4). This step is a step of estimating a segment shape including at least one of a curvature, a bend amount, a bend direction and a bend shape of each segment 56, based on the segmentation information acquired in the step S2 and the first curvature information acquired in the step S3.

In a concrete example of the estimation, as illustrated in FIG. 5 to FIG. 9 and equation (1) and equation (2), second curvature information, which is a curvature (1/R), a bend amount (θ) or a bend direction (α) of each segment 56, is calculated from the first curvature information at the sensing parts 46. Based on the second curvature information, the bend shape of each segment 56, in particular, the shape in the case in which an arc is assumed, is estimated.

Next, the shape estimation unit 24 executes shape estimation of the target by segment connection (step S5). This step is a step of connecting the neighboring segments 56, based on the segment shapes estimated in the step S4, and estimating the shape in a predetermined range of the bend member that is the target, such as the insertion section 26 of the bend mechanism of the endoscope 12 or the like.

In this step, the connection as illustrated in FIG. 12 is executed. The connection is executed according to the following rules:
1) The segments are continuously connected at connection parts between the segments,
2) The directions (tangent directions) of the respective segment end portions coincide at the connection parts between the segments, and
3) The segments are connected at the connection parts between the segments, with no twist or rotation of the segments 56.

The bend shape thus connected is set to be the bend shape of the predetermined range of the bend member that is the target.

Thereafter, the shape estimation unit 24 outputs to the display unit 55 the estimated bend shape of the predetermined range of the bend member that is the target (step S6). Incidentally, the mode of display to the display unit 55 is not specified here.

Then, the shape estimation unit 24 determines whether the shape estimation has ended or not (step S7). In this step, it is confirmed whether the shape estimation is continued or not. If the shape estimation is continued, the process returns to the step S3, and the step S3 to step S6 are repeated. If it is determined that the shape estimation is finished, the process exits the repetition of step S3 to step S6, and the process is terminated.

In the meantime, even when the shape estimation is finished, for example, if the bend shape estimation system 50 is powered on once again or the program is re-executed, the execution of the process from the step S2 may be resumed.

By adopting this method, the shape estimation in the predetermined range of the bend member that is the measurement target can be executed simply and efficiently, with no unnecessary process.

Although the present invention has been described above based on one embodiment, the present invention is not limited to the above-described embodiment, and, needless to say, various modifications and applications can be made without departing from the spirit of the invention.

For example, the display unit 55 may not be included in, but may be disposed outside, the bend shape estimation system 50. Such an external display unit may serve also as the display unit 16 of the endoscope system 10 that is the tubular insertion system to which this bend shape estimation system is applied. When the display unit 55 is disposed inside or outside the bend shape estimation system 50, the estimation result is directly output from the shape estimation unit 24 to the display unit 55. On the other hand, when the external display unit serves also as the display unit 16 of the endoscope system 10, the estimation result may indirectly be output to the display unit 16 via the control device 22 of the endoscope system 10. Thereby, an endoscopic observation image and the bend shape in the predetermined range of the insertion section 26 can be display in juxtaposition, or can be switched and displayed.

Additional advantages and modifications will readily occur to those skilled in the art. Therefore, the invention in its broader aspects is not limited to the specific details, representative devices, and illustrated examples shown and described herein. Accordingly, various modifications may be made without departing from the spirit or scope of the general inventive concept as defined by the appended claims and their equivalents.

What is claimed is:

1. A bend shape estimation system comprising:
a shape estimation unit, wherein
when a predetermined range of a bend member is divided into a plurality of segments which neighbor in order in a longitudinal direction and are estimation units each having at least information of a length, a curvature and a direction for estimating a bend shape of the bend member, the shape estimation unit includes:
a segment shape estimation unit configured to estimate a shape of each of the segments by using segment information including at least one piece of curvature information with respect to each segment; and
a bend member shape estimation unit configured to estimate the bend shape in the predetermined range of the bend member by connecting end portions of each two neighboring segments, such that tangent directions of end portions of the estimated shapes of the two neighboring segments coincide and directions around the tangent directions coincide.

2. The bend shape estimation system of claim 1, wherein
each of all segments is provided with two or more sensing parts for detecting curvatures or bend amounts in mutually different directions, which are perpendicular to the longitudinal direction of the bend member, and
the shape estimation unit is configured to estimate any one of the curvature, bend amount and bend shape of each segment.

3. The bend shape estimation system of claim 2, wherein
the different directions are two directions which differ by 90° in an xy plane defined by an x-axis and a y-axis in the sensing part, and
the sensing parts for detecting curvatures in the two directions are included, one by one, in each of the segments.

4. The bend shape estimation system of claim 2, wherein
a direction of the curvature detected at the sensing part, which is represented by using an x-axis and a y-axis in the sensing part, is set as a direction on an xy coordinate system, and
a combination of directions on the xy coordinate system, in which the directions on the xy coordinate systems are combined with respect to all sensing parts in the segments, is a combination of identical directions in all segments.

5. The bend shape estimation system of claim 2, wherein
a plurality of the sensing parts in each of the segments, which detect the curvatures in the different directions, are configured to detect curvatures in mutually different directions, and
the sensing parts are located at a substantially identical position in the longitudinal direction of the bend member.

6. The bend shape estimation system of claim 1, wherein
each of all segments is provided with one or more sensing parts for detecting curvatures or bend amounts in directions perpendicular to the longitudinal direction of the bend member, and
with respect to sensing parts in each segment,
the sensing parts are all disposed at a substantially identical position, and
a boundary between each neighboring segments is a midpoint in the longitudinal direction between the substantially identical positions at which the sensing parts in the respective segments are located.

7. The bend shape estimation system of claim 6, wherein
a position in the longitudinal direction of the bend member, at which bend characteristics sharply change, is set as a second segment boundary, and
when the boundary of the segments that is the midpoint between the sensing parts, which are closest in the longitudinal direction on both sides of the second segment boundary, is located at a position different from the second segment boundary in the longitudinal direction,
the second segment boundary is used as the boundary of the segments in place of the boundary of the segments that is the midpoint.

8. The bend shape estimation system of claim 7, wherein
the bend member includes:
an active bend portion which is located on a distal-end side of the bend member and is bendable by an operation; and
a passive bend portion which is located on a proximal side of the bend member and bends passively by force from an outside, and
a connection position between the active bend portion and the passive bend portion is set as the second segment boundary.

9. The bend shape estimation system of claim 7, wherein
the predetermined range of the bend member has flexibility,
a rigid portion, which does not bend, is present within the predetermined range having the flexibility, and
both ends in the longitudinal direction of the rigid portion are set as the second segment boundaries.

10. The bend shape estimation system of claim 1, wherein
when a predetermined bending moment is applied to the bend member,
a boundary of two neighboring segments is located at a position where a bend amount from a sensing part in one segment of the two neighboring segments to a sensing part in the other segment is halved.

11. The bend shape estimation system of claim 1, further comprising:
a shape sensor including a plurality of sensing parts disposed in the respective segments and configured to detect first curvature information which is curvature components or bend amount components with respect to predetermined bend directions,
wherein the shape estimation unit is configured to derive, from the first curvature information, second curvature information which is a bend direction and a curvature or a bend amount of each of the segments, and to estimate a bend shape of each segment, based on the second curvature information.

12. The bend shape estimation system of claim 11, wherein
the shape sensor is a fiber sensor including:
at least one optical fiber with flexibility including a plurality of sensing parts for detecting a curvature and a bend direction;
a light source configured to supply detection light to the optical fiber;
an optical detector which is capable of detecting optical characteristics corresponding to a curvature at each of the plurality of sensing parts, based on characteristics of light traveling via the plurality of sensing parts; and
a bend calculator configured to calculate the curvature at each of the plurality of sensing parts, which is the segment information, based on the optical characteristics.

13. The bend shape estimation system of claim 1, wherein
the shape estimation unit is configured to estimate the shape of each segment, assuming that the shape of each segment is arcuate.

14. A tubular insertion system comprising:
an insertion section with flexibility configured to be inserted in a lumen of a subject and to perform a predetermined work;
the bend shape estimation system of claim 1 for estimating a bend shape of the insertion section, the insertion section being the bend member; and
a shape sensor including, in each segment, at least one sensing part for acquiring the segment information of each segment.

15. The tubular insertion system of claim 14, wherein
the insertion section includes: at a distal end thereof, an active bend portion which is capable of a bending operation and has a large bend amount; and on a proximal side thereof, a passive bend portion which neighbors the active end portion, has flexibility, and has a smaller bend amount per unit length, compared to the active bend portion,
a contact point between the active bend portion and the passive bend portion is set as a second segment boundary,
segments, which are fine and substantially equal in width, are disposed in the longitudinal direction in the active bend portion, and
segments, which are longer than the segments of the active bend portion and are substantially equal in width, are disposed in the longitudinal direction in the passive bend portion.

16. A bend shape estimation method of a bend member in a bend shape estimation system configured such that a predetermined range with flexibility of the bend member is divided into a plurality of segments which neighbor in order in a longitudinal direction, a shape of each of the segments is estimated by using segment information including at least one piece of curvature information with respect to each segment, and a shape of the bend member is estimated based on each estimated segment shape, the method comprising:
segmenting the predetermined range of the bend member into a plurality of segments neighboring in order in a longitudinal direction of the bend member, such that at least one of a plurality of sensing parts, which are disposed along the longitudinal direction of the bend member, is included in each of the segments;
acquiring segmentation information which is information necessary for shape estimation, other than curvature information, the segmentation information including a disposition and a length of each segment;
acquiring segment information which includes the curvature information detected at the plurality of sensing parts;
estimating a segment shape including at least one of a curvature, a bend amount, a bend direction and a bend shape of each segment, based on the segmentation information and the segment information;
connecting neighboring segments whose segment shapes were estimated and estimating a shape of an entirety of the predetermined range of the bend member;
confirming whether the shape estimation is continued or not;
repeating the acquiring segment information, the estimating segment shape and the connecting neighboring segments and estimating the shape if a result of the confirming is that the shape estimation is continued; and
exiting the repeating and finishing the shape estimation if the result of the confirming is that the shape estimation is finished.
